(19) United States
(12) Patent Application Publication
(10) Pub. No.: US 2024/0074665 A1
(43) Pub. Date: Mar. 7, 2024

(54) CORE TEMPERATURE SENSING WITH WEARABLE ELECTRONIC DEVICE

(71) Applicant: Apple Inc., Cupertino, CA (US)

(72) Inventors: Daniel J. Hiemstra, San Francisco, CA (US); Jeffrey W. Buchholz, Santa Cruz, CA (US); Xiaofan Niu, San Jose, CA (US); James C. Clements, Campbell, CA (US); Wei Lin, Santa Clara, CA (US); Habib S. Karaki, Sunnyvale, CA (US); Paul Mansky, San Francisco, CA (US); Boyi Fu, San Jose, CA (US); Yanfeng Chen, San Ramon, CA (US); Edmilson Besseler, Foster City, CA (US)

(21) Appl. No.: 18/147,628

(22) Filed: Dec. 28, 2022

Related U.S. Application Data
(60) Provisional application No. 63/374,317, filed on Sep. 1, 2022.

(51) Int. Cl. A61B 5/01; A61B 5/00; G01K 13/20

(52) U.S. Cl. CPC A61B 5/01; A61B 5/681; A61B 5/7203; A61B 5/7246; G01K 13/20; A61B 2562/0271; A61B 2562/12

(57) ABSTRACT

An electronic device includes a housing defining an internal volume, a front opening, and a rear opening. The electronic device can include a display component disposed at the front opening and a rear cover disposed at the rear opening. A logic board can be disposed in the internal volume. The device can also include a thin film thermopile including a cold junction bonded to the logic board and a hot junction bonded to the rear cover.

### CROSS-REFERENCE TO RELATED APPLICATION(S)

This claims priority to U.S. Provisional Patent Application No. 63/374,317, filed 1 Sep. 2022, and entitled “CORE TEMPERATURE SENSING WITH WEARABLE ELECTRONIC DEVICE,” the entire disclosure of which is hereby incorporated by reference.

### FIELD

The examples described in the present disclosure relate generally to temperature sensing devices. More particularly, the examples described in the present disclosure relate to a wearable electronic device for sensing core body temperature.

### BACKGROUND

Recent advances in computing technology have enabled miniaturized, wearable electronic devices capable of multi-functionality. Users can browse the internet and send emails on mobile phones or record exercises and measure burned calories with electronic watches. Wearable electronic devices can be secured against the skin or body of a user and include sensors to detect various health related conditions, for example a user's heart rate or blood-oxygen levels. While it would be advantageous to track a user's body temperature suing such devices, body temperature sensing with wearable devices presents a number of challenges. For example, the temperature of the device and the environment in which it is used can change from moment to moment during use. Core temperatures can be measured with medical thermometers with access to a person's mouth or ear cavity, but these methods are invasive and not compatible with typical wearable electronic devices such as electronic watches.

Therefore, what is needed in the art are wearable devices for detecting core body temperature while worn in the normal course of use and which account for variable use and operational conditions.

### SUMMARY

In at least one example of the present disclosure, an electronic device includes a housing defining an internal volume, a front opening, and a rear opening. The electronic device can include a display component disposed at the front opening, and a rear cover disposed at the rear opening. A logic board can be disposed in the internal volume. The device can also include a thin film thermopile including a cold junction bonded to the logic board and a hot junction bonded to the rear cover.

In one example, the logic board includes an upper molded layer and the cold junction is bonded to the upper molded layer. In one example, the logic board comprises an electronic interconnect extending through the molded layer and contacting the cold junction. In one example, the cold junction is bonded via an isotropic conductive film (ICF). In one example, the hot junction is bonded via a thermal epoxy. In one example, at least a portion of the thin film thermopile is routed within a flex. In one example, the electronic device further includes a processor electrically coupled to the thin film thermopile via the logic board, the processor configured to determine a core temperature of a user contacting the rear cover based on a temperature difference between the cold junction and the hot junction. In one example, the temperature difference is used to generate a heat flux correction factor. In one example, the processor executes an algorithm stored on a memory component, the algorithm taking into account the heat flux correction factor to determine the core temperature.

In at least one example of the present disclosure, a wearable electronic device includes a housing sidewall defining an internal volume, and a first strap retention feature opposite the first strap retention feature, a rear cover, and a core temperature sensing assembly. The core temperature sensing assembly can include a logic board disposed in the internal volume, and a temperature sensor including a first junction bonded to the logic board and a second junction bonded to the rear cover.

In one example, the temperature sensor is configured to sense a temperature difference between the first junction and the second junction. In one example, the temperature sensor includes a thin film thermopile. In one example, the rear cover is configured to press against a body of a user when the user dons the wearable electronic device via a retention strap connected to the first and second strap retention features. In one example, the rear cover defines an external rear surface of the wearable electronic device. In one example, the wearable electronic device further includes a display assembly having a transparent cover, the transparent cover defining an external front surface of the wearable electronic device opposite the external rear surface.

In at least one example of the present disclosure, a method of measuring a core body temperature with a wearable electronic device can include bonding a first junction of a thin film thermopile to a logic board disposed in the wearable electronic device, bonding a second junction of the thin film thermopile to a rear cover of the wearable electronic device, generating a heat flux correction factor based on a temperature difference between the first junction and the second junction, and calculating the core body temperature based on an algorithm taking into account the heat flux correction factor.

In one example, the method further includes pressing the rear cover against a body before generating the heat flux correction factor. In one example, the second junction measures a temperature of the rear cover. In one example, the algorithm correlates the temperature of the rear cover to a surface temperature of the body. In one example, the algorithm correlates the surface temperature of the body to the core body temperature.

### BRIEF DESCRIPTION OF THE DRAWINGS

The disclosure will be readily understood by the following detailed description in conjunction with the accompanying drawings, wherein like reference numerals designate like structural elements, and in which:

FIG. 1A shows a perspective view of an example of a wearable electronic device;

FIG. 1B shows a top perspective view of a portion thereof;

FIG. 1C shows a bottom perspective view thereof;

FIG. 2 shows an exploded view of an example of a wearable electronic device;

FIG. 3 shows a side view of an example of a wearable electronic device;

FIG. 4 shows a side cross-sectional view of an example of a wearable electronic device;

FIG. 5 shows a circuit diagram of a system for measuring core body temperature using a wearable electronic device;

FIG. 6A shows a perspective view of a portion of an example of a wearable electronic device;

FIG. 6B shows a partial cross-sectional view thereof;

FIG. 6C shows another partial cross-sectional view thereof;

FIG. 7A shows a partial top view of an example of a wearable electronic device;

FIG. 7B shows a partial close-up view thereof;

FIG. 8A shows a top view of a portion of an example of a wearable electronic device;

FIG. 8B shows a perspective view thereof including a thermopile;

FIG. 9 illustrates an example of a method of manufacturing a printed circuit board (PCB) with conduction pins extending through an upper molded layer;

FIG. 10 shows an example of a method of manufacturing a PCB with conduction pins extending through an upper molded layer;

FIG. 11 shows an example of a method of manufacturing a PCB with conduction pins extending through an upper molded layer;

FIG. 12A shows an example of a method of manufacturing a portion of a wearable electronic device;

FIG. 12B shows a partial cross sectional view of the wearable electronic device shown in FIG. 12A;

FIG. 12C shows another partial cross-sectional view thereof; and

FIG. 12D shows an example of an assembly used in the manufacturing method used in FIG. 12A.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The following disclosure relates generally to temperature sensing devices. More particularly, the examples described in the present disclosure relate to wearable electronic devices for sensing core body temperature. In a particular example, an electronic device includes a housing defining an internal volume, a front opening, and a rear opening. The electronic device can include a display component disposed at the front opening and a rear cover disposed at the rear opening. A logic board can be disposed in the internal volume. The device can also include a thin film thermopile including a cold junction bonded to the logic board and a hot junction bonded to the rear cover.

Because wearable electronic devices are in contact with the user's body during use, it can be advantageous to use such a device for non-invasive measurement of the user's core body temperature based on a measurement of the user's surface skin temperature where the device makes contact. However, the temperature of the device and the environment in which it is used can change from moment to moment during use such that detecting the user's core temperature with a wearable device can be challenging. Devices described herein can overcome these challenges by measuring a temperature difference between two locations within the device, with one of the locations being close to the portion of the device contacting the user's skin, and calculating a heat flux correction factor based on heat flux from the skin through the device.

The heat flux correction factor can be used in one or more algorithms, as executed by a processor of the device, to determine a surface temperature of the skin. The surface temperature can then be used to extrapolate a user's core temperature. In examples disclosed herein, the temperature difference between two locations within the device can be measured using a thin film thermopile having a hot junction at a first location (e.g., a location near the user's skin) and a cold junction at a second location (e.g., on a logic board within the device). In at least one example, as noted above, the device can include a rear cover configured to press against the skin of the user when the device is donned. The first location can include the rear cover of the device where the hot junction of the thermopile can be bonded.

The thin film thermopile can be routed from the first location to the second location for measuring the temperature difference in any shape, path, or configuration between various components within the device. The thermopile can be bent to extend between adjacent components and around corners to directly contact the logic board at one end and the rear cover at the other end. In this way, the thermopile can save space within the device for more compact designs. In addition, thin film thermopiles described herein can be advantageous to avoid drift inherent in multi-sensor configurations where a first sensor is located at the first location and a second, separate sensor is located at the second location. Rather, the single thermopile can measure a temperature difference between its hot and cold junctions located at different positions within the device. In at least one example, the thin film thermopile can be routed from one location to another such that no heat generating components are disposed between the hot and cold junctions thereof. In this way, the heat flux of the device between the junctions is minimized or eliminated to simplify the core temperature calculation and algorithms.

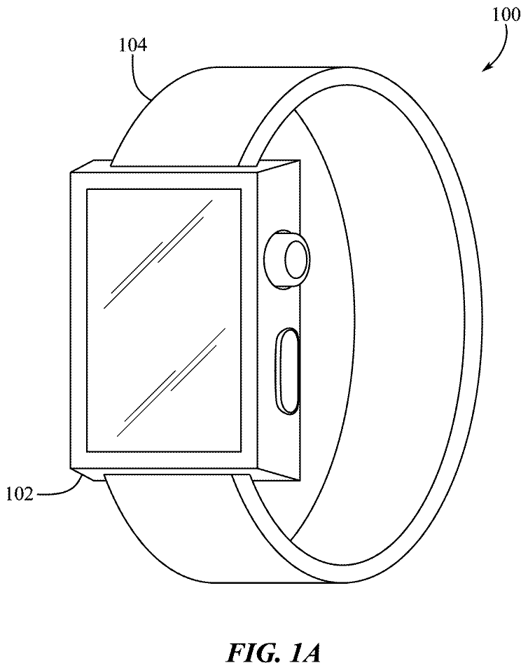

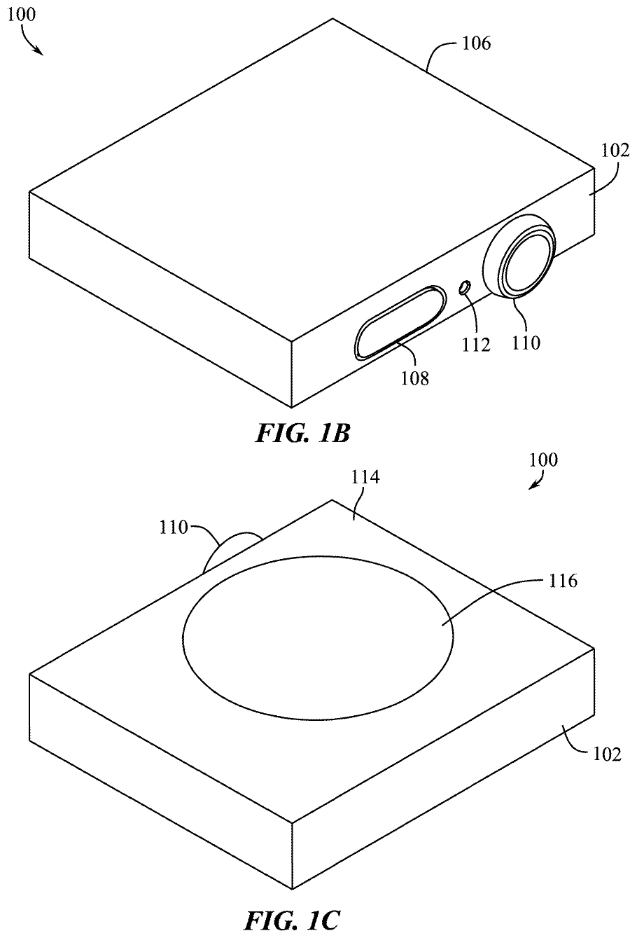

These and other embodiments are discussed below with reference to FIGS. 1-12B. However, those skilled in the art will readily appreciate that the detailed description given herein with respect to these Figures is for explanatory purposes only and should not be construed as limiting. Furthermore, as used herein, a system, a method, an article, a component, a feature, or a sub-feature comprising at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

FIG. 1A shows an example of a wearable electronic device 100, which can also be referred to herein as an electronic device 100 or device 100. The electronic device shown in FIG. 1A is a watch, such as a smartwatch. The smartwatch of FIG. 1A is merely one representative example of a device that can be used in conjunction with the systems and methods disclosed herein. Electronic device 100 can correspond to any form of wearable electronic device, a portable media player, a media storage device, a portable digital assistant (“PDA”), a tablet computer, a computer, a mobile communication device, a GPS unit, a remote control device, or other electronic device. The electronic device 100 can be referred to as an electronic device, or a consumer device. In some examples, the electronic device 100 can include a housing 102 that can carry operational components, for example, in an internal volume at least partially defined by the housing. The electronic device 100 can also include a strap 104, or other retaining component that can secured the device 100 to a body of a user as desired. Further details of the electronic device are provided below with reference to FIG. 1B.

FIG. 1B illustrates the electronic device 100, for example a smartwatch, that can be substantially similar to and can include some or all of the features of the devices described herein, including the electronic device 100 shown in FIG. 1A but without the strap 104. The device 100 can include a housing 102, and a display assembly 106 attached to the housing 102. The housing 102 can substantially define at least a portion of an exterior surface of the device 100.

The display assembly 106 can include a glass, a plastic, or any other substantially transparent cover defining a front external surface of the device 100. The display assembly 106 can include multiple layers, with each layer providing a unique function, as described herein. Accordingly, the display assembly 106 can be, or can be a part of, an interface component. The display assembly 106 can define a front exterior surface of the device 100 and, as described herein, this exterior surface can be considered an interface surface. In some examples, the interface surface defined by display assembly 106 can receive inputs, such as touch inputs, from a user.

In some examples, the housing 102 can be a substantially continuous or unitary component and can define one or more openings to receive components of the electronic device 100. In some examples, the device 100 can include input components such as one or more buttons 108 and/or a crown 110 that can be disposed in the openings. In some examples, a material can be disposed between the buttons 108 and/or crown 110 and the housing 102 to provide an airtight and/or watertight seal at the locations of the openings. The housing 102 can also define one or more openings or apertures, such as aperture 112 that can allow for sound to pass into or out of the internal volume defined by the housing 102. For example, the aperture 112 can be in communication with a microphone component disposed in the internal volume. In some examples, the housing 102 can define or include a feature, such as an indentation to removably couple the housing 102 and a strap or retaining component.

FIG. 1C shows a bottom perspective view of the electronic device 100. The device 100 can include a back side 114 that can be attached to the housing 102, for example, opposite the display assembly 106. The back side 114 can include ceramic, plastic, metal, or combinations thereof. In some examples, the back side 114 can include a rear cover 116 defining an external rear surface of the device 100. The rear cover 116 can be configured to press against the body of a user when the user dons the device 100. In at least one example, the rear cover 116 can include an at least partially electromagnetically transparent material. The electromagnetically transparent material can be transparent to any desired wavelengths of electromagnetic radiation, such as visible light, infrared light, radio waves, or combinations thereof. In some examples, the electromagnetically transparent material of the rear cover 116 can allow sensors and/or emitters disposed in the housing 102 to communicate with the external environment.

Together, the housing 102, display assembly 106, and the back side 114 including the rear cover 116, can substantially define an internal volume and an external surface of the device 100. For example, the rear cover 116 can define an external rear surface of the device 100 and the display assembly 106 can define the external front surface of the device 100 opposite the external rear surface. In particular, a transparent cover or layer of the display assembly 106 noted above can define the external front surface of the device 100 opposite the external rear surface defined by the rear cover 116.

Any of the features, components, and/or parts, including the arrangements and configurations thereof shown in FIGS. 1A-1C can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 1A-1C.

As noted above, portable and wearable electronic devices can be designed to be used in many different environments and during any kind of activity throughout a user's day. For example, wearable electronic watches, headphones, and phones can be carried by a user during exercise, sleep, driving, biking, hiking, swimming, diving, outside in the rain, outside in the sun, and so forth. Wearable electronic devices described herein are configured to withstand the varied and often harsh conditions of various environments, including changing environments and wet environments. Wet environments can include wearing devices in the rain or when submerged during bating or swimming, for example.

Examples of electronic devices disclosed herein include components, features, arrangements, and configurations that resists damage and corrosion due to exposure to moisture. Some aspects of devices described herein can include gaps between components through which moisture, water, or other fluids could enter. The gaps may be present for aesthetic purposes or for functional purposes. However, one or more components, including epoxy seals, insulating materials and frames, and other components of devices described herein can be configured to prevent such moisture from entering into the internal volume of the device where sensitive electronic component could be damaged thereby.

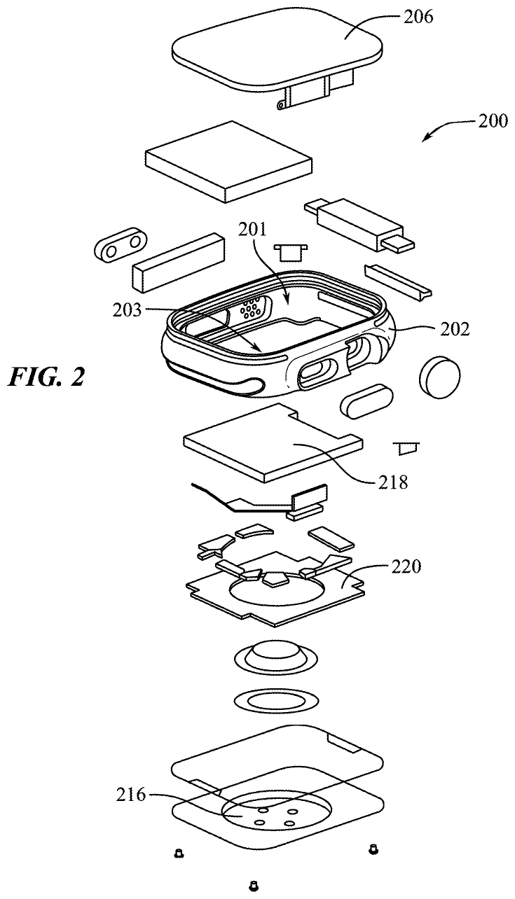

FIG. 2 illustrates an exploded view of another example of an electronic device 200, which can also be a portion of a wearable electronic watch or other wearable electronic device. Device 200 includes a display assembly 206, housing 202, and rear cover 216. In addition, the exploded view of FIG. 2A illustrates various internal components that may be disposed within an internal volume defined by the housing 202, rear cover 216, and display assembly 206. For example, the device 200 can include one or more printed circuit boards (PCBs) 218, otherwise referred to herein as “logic boards,” and one or more antenna components 220, electrical connectors and flexes, buttons, seals, gaskets, memory components, processors, sensors, dials, batteries, and so forth.

In at least one example, the housing 202 can form one or more sidewalls, as shown in FIG. 2, defining a front opening 201 and a rear opening 203. The display component or assembly 206, including a display layer and a transparent cover, can be disposed at or in the front opening 201. The rear cover 216 can be disposed at or in the rear opening 203.

Any of the features, components, and/or parts, including the arrangements and configurations thereof shown in FIG. 2 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 2.

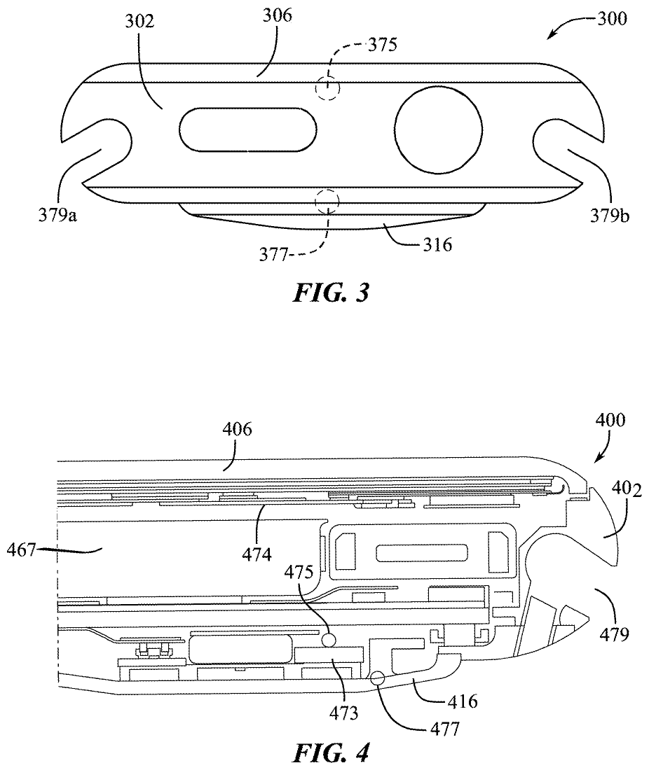

FIG. 3 illustrates an example of an electronic device 300, for example a wearable electronic watch device 300. In at least one example, the device 300 can include a housing 302 defining front and rear openings, with a display component 306 disposed at the front opening and a rear cover 316 disposed at the rear opening. The device 300 of FIG. 3 can also include a first strap retention feature 379a and a second strap retention feature 371b opposite the first strap retention feature 379a. In at least one example, the strap retention features 379a and 379b can be defined by the housing 302 and be configured for securing a strap to the device 300. When a strap is connected to the device 300 via the strap retention features 379, the device 300 can be configured to be worn by a user, for example on the wrist of a user, with the strap securing the rear cover 314 against the skin of the user.

In such an example, the device 300 can be configured to detect a wrist or skin temperature of the user and extrapolate or detect/measure the user's core temperature. In order to do this, in at least one example, the device 300 can include one or more temperature sensors on or within the device 300. In at least one example, the one or more temperature sensors can configured to detect temperatures within the device 300 at a first location 377 and a second location 375. The first and second locations 377, 375 shown in the example of FIG. 3 are exemplary and only for purposes of illustration and explanation. Other examples of devices can include sensors measuring/detecting temperature within the device 300 at different locations.

In the example shown in FIG. 3, the first location 377 can be located at, near, or adjacent the rear cover 316, as indicated by the lower dotted circle shown in FIG. 3. This first location 377 can also referred to as the bottom or lower side of the device 300. The second temperature sensing location 375 can be separated from the first location 375 within the device 300. In the example shown in FIG. 3, the second location 375 can be at, near, or adjacent the display component 306 on an opposite side from the first temperature sensor.

In at least one example, a processor (not shown in FIG. 3 but disposed inside the device 300) can be electrically connected to the one or more sensors detecting temperatures at the first and second locations 377, 375. The processor can be electrically coupled to a memory component storing electronic instructions that, when executed by the processor, causes the processor to determine a core temperature of a user based on a temperature difference between the first and second locations 377, 375.

Any of the features, components, and/or parts, including the arrangements and configurations thereof shown in FIG. 3 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 3.

FIG. 4 illustrates a partial cross-sectional view of a device 400, which can be similar to the device 300 shown in FIG. 3, to illustrate various internal components thereof. As shown, the device 400 can include a housing 402 defining front and rear openings and an internal volume, with a display component 406 disposed at the front opening and a rear cover 416 disposed at the rear opening. The internal components can include various processors, batteries, microphones, speakers, wires and electrical flexes, antennas, display components, and so forth. In addition, the internal components of the device 400 can include a logic board 473 disposed near, adjacent, and above the rear cover 416. In at least one example, the logic board 473 can be adhered to the rear cover 416.

As shown in the cross-sectional view of FIG. 4, the first temperature sensor location 477 can be disposed on, against, or adjacent the rear cover 416 and the second temperature sensor location 475 can be disposed on, against, or adjacent the logic board. In at least one example, one or more other electronic components, including heat generating electronic components such as a battery 467, can be disposed in the device 400. However, in at least one example, the first and second temperature sensing locations 477, 475 can be disposed such that no heat generating components are disposed between the first and second temperature sensing locations 477, 475.

In at least one example, the device 400 can include a first temperature sensor located at the first temperature sensing location 477 and a second temperature sensor located at the second temperature sensing location 475. The two temperature sensors can be in electrical communication with a processor or other electronic component to determine a temperature difference between the first and second locations 477, 475 as detected by the sensors. In one example, the device 400 can include a single temperature sensor detecting the temperature difference between the first and second locations 477, 475 within the device 400. In example having a single temperature sensor detecting the temperature difference between the first and second locations 477, 475 within the device 400, the temperature sensor can include a first junction located at the first temperature sensing location 477 and a second junction located at the second temperature sensing location 475 and configured to sense the temperature difference between the first junction and the second junction.

In at least one example, the device 400 can include one or more processors in electrical communication with the temperature sensor(s) detecting the temperature difference between the first and second temperature sensing locations 477, 475. The one or more processors can determine the user's core temperature from the measured temperature difference with one or more algorithms, taking into account the temperature difference, to extrapolate a core temperature of the user donning the device with the rear cover 416 pressed against the skin. The algorithm can also take into account a thermal path through the rear cover 416, between the skin and the first location 477 within the device 400, to extrapolate a surface temperature of the user and then extrapolate a core temperature based on the extrapolated surface temperature. The algorithm can also take into account the thermal path variables between the first and second locations 477, 475. In examples where no heat generating components are disposed between the first and second locations 477, 475, and/or in examples where a single temperature sensor measures the temperature difference between the first and second locations 477, 475, the variable of the thermal path between the first and second locations 477, 475 can be minimized to increase accuracy and consistency of the measurement and core temperature determination.

Any of the features, components, and/or parts, including the arrangements and configurations thereof shown in FIG. 4 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 4.

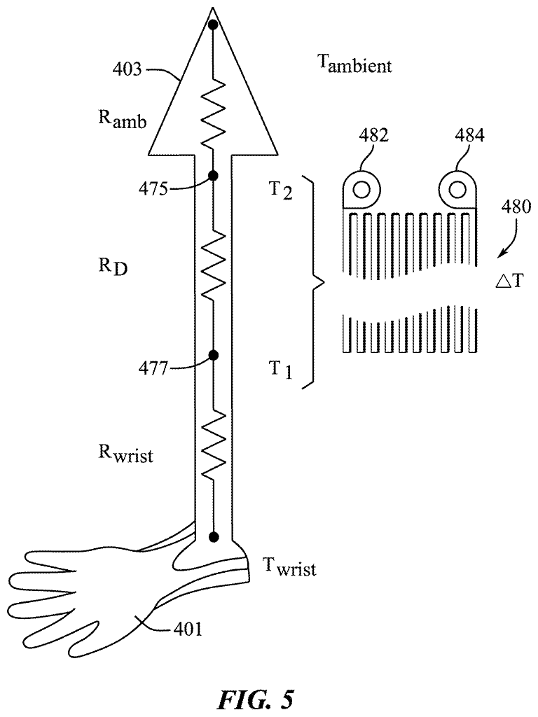

FIG. 5 illustrates a circuit diagram equivalent to a wearable electronic device donned by a user, for example on or against a wrist 401 of a user. The device can be similar to the devices 100, 200, 300, and 400 shown in FIGS. 1-4. The illustrated diagram of FIG. 5 shows temperatures T₁ and T₂, which can equate to the first and second temperature sensing locations 477, 475 shown in FIG. 4. The difference in T₁ and T₂ can be referred to as ΔT. Other temperatures of the system are also shown, including T_{wrist} representing the temperature of the wrist 401 and T_{ambient} representing the temperature of the ambient environment external to the device and wrist 401. The circuit diagram of FIG. 5 also illustrates a heat transfer path from the user's wrist 401 through the device and out to an ambient environment, which can be modeled as a series of resistances illustrated by resistors R_{wrist} (representing a resistance of the user's wrist), R_{D} (representing the resistance of the device between locations where T₁ and T₂ are measured), and R_{amb} (representing a resistance of the ambient environment). The heat flow through the system from the wrist 401 and through the device into the ambient environment is indicated at arrow 403.

Using the modeled circuit diagram of heat flow from the wrist 401 and through the device as shown in FIG. 4 and the diagram in FIG. 5, one or more algorithms can be used to determine the core temperature of the user. Using Fourier's Law:

Q=−(1/R)(ΔT)

where Q=heat flux, R=resistance, and T=temperature. Assuming a constant heat flux (Q₁=Q₂) then T_{wrist} can be modeled or calculated as follows:

T_{wrist}=T₁+(Rₒ/R₁₋₂)(ΔT)

Using the algorithm shown above, another algorithm can extrapolate a user's core temperature from the determined surface temperature T_{wrist} of the user's wrist 401. In addition, one or more of the algorithms can use the measured ΔT to generate a heat flux correction factor. The heat flux correction factor can be taken into account to determine the core temperature based on T_{wrist} and one or more other algorithms, including the algorithms shown above.

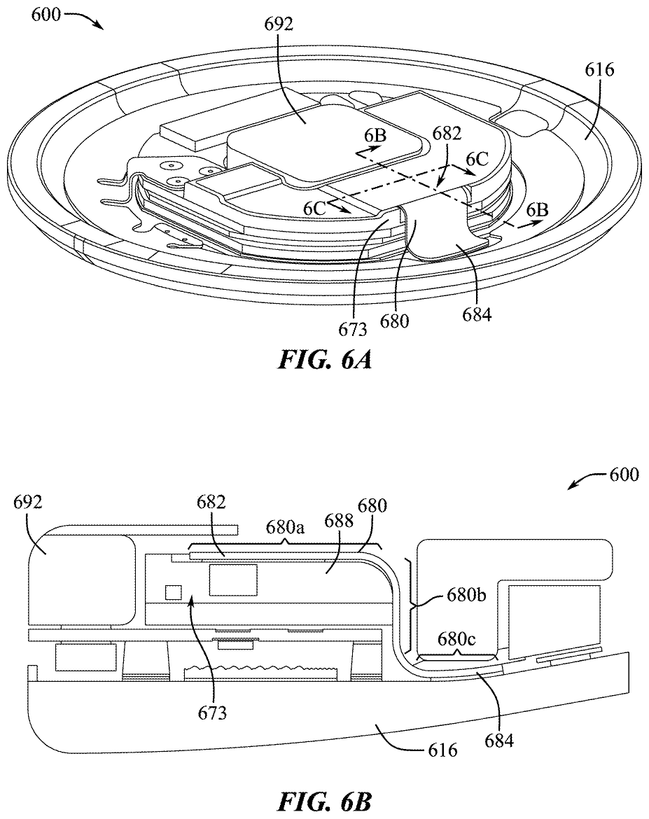

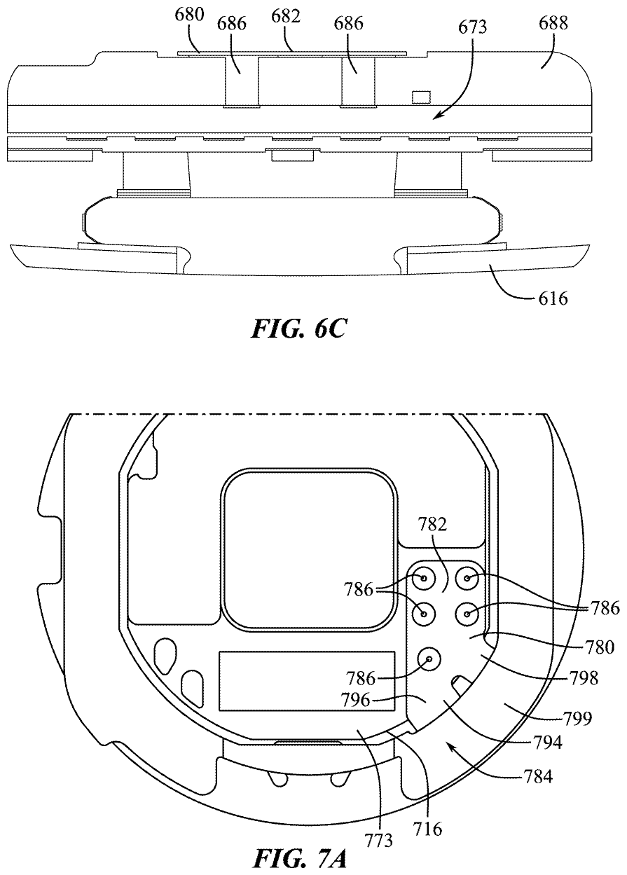

In one example, ΔT can be determined by the difference between two sensor measurements, with one at the first location location 477 and another at the second location 475. In one or more other examples, ΔT can be determined by a single sensor having opposing junctions at the two locations 477, 475. For example, the devices described herein can include a thermopile having a hot junction 482 on the rear cover 416 at the first location 477 and a cold junction 484 on a logic board (or “PCB”) located at the second location 475. A thermopile, such as a thin-film thermopile routed to extend from the first location 477 to the second location 475 can directly measure ΔT to remove drift error between two separate sensors. FIGS. 6A and 6B show an example of a device 600 incorporating such a thermopile.

Any of the features, components, and/or parts, including the arrangements and configurations thereof shown in FIG. 5 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 5.

FIGS. 6A-6C illustrates an example of a portion of a device 600 including a rear cover 616 defining an external surface and an internal volume. The device 600 also includes a logic board 673 disposed in the internal volume. The device 600 can also include a thermopile 680, for example a thin-film thermopile including a cold junction 682 and a hot junction 684. In one example, the cold junction 682 can be bonded to the logic board 673 and the hot junction 682 can be bonded to the rear cover 616. The rear cover 616 is configured to be pressed against or make contact with the skin of a user.

In one example, the logic board 673 and the thermopile 680 can be referred to as a core temperature sensing assembly. In at least one example, the logic board 673 can include an upper molded layer or portion 688 and the cold junction 682 can be bonded to the molded layer 688. In at least one example, as shown in the partial cross-sectional view of FIG. 6B, the thing film thermopile 680 can include three portions 680a, 680b, and 680c. The first portion 680a can include the cold junction 682 disposed above and bonded to the logic board 673. The third portion 680c can include the hot junction 684 and be disposed above and bonded onto the rear cover 616. The second portion 680b of the thermopile 680 can extend from the first portion 680a and 680c. In at least one example, the first portion 680a is disposed elevationally above the third portion 680c relative to the rear cover 616. In such an example, the second portion 680b of the thermopile 680 can span the elevation difference, for example vertically from the first portion 680a to the third portion 680c.

In at least one example, the second portion 680b of the thermopile 680 can be disposed between the logic board 673, including the upper molded layer 688 thereof, and an adjacent component 690 of the device 600. The adjacent component 690 can be any other component of the device 600 disposed in the internal volume thereof. Examples of other components 690 can include batteries, memory components, PCBs, wires, brackets, fasteners, electrical flexes, antennas, lights, sensors, receivers, speakers, and so forth. In at least one example, as shown in FIG. 6B, the thermopile 680 can be routed from the logic board 673 at the cold junction 682 to the rear cover 616 at the hot junction 684 between the logic board 673 (including the molded layer 688) and the adjacent component 690 with the second portion 680b extending vertically or at least non-parallel to the first and third portions 680a, 680c of the thermopile 680 between the logic board 673 (including the molded layer 688) and the adjacent component 690. In this way, the thermopile 680 can form a tortuous route between various components of the device 600 to preserve space in the internal volume thereof.

In other examples, thin film thermopile temperature sensors can be routed in other irregular pathways to accommodate other components of the device 600 to save space. Thin film thermopiles can be intricately wound through tight spaces and tortuous, curvilinear, and irregular geometries to reach a between a first junction at a first temperature sensing location to a second junction at a second temperature sensing location based on design and system needs. Thin film thermopiles can include widths, lengths, and dimensions the same or different from those shown in the present disclosure. It will be appreciated that the geometry, pathway, dimensions, and general configuration of thin film thermopiles can be customized and designed differently for different devices having different temperature sensing requirements and components. Thermopiles can be used to maintain accurate AT measurements while minimizing a volume taken up by the sensor measuring/detecting the temperatures. In at least one example, the thermopile 680 can be less than 0.5 mm thick, for example less than 0.3 mm thick. In one example, the thermopiles 680 disclosed herein can be between about 0.1 and about 0.15 mm thick.

In at least one example, as shown in the partial cross-sectional view of FIG. 6C, the logic board 673 can include one or more electronic interconnects 686 extending through the molded layer 688 to contact and electronically couple the cold junction 682 of the thermopile 680 with one or more circuit elements or components on the logic board 673 below the molded layer 688. In at least one example, the cold junction 682 of the thermopile 680 can be bonded to the electronic interconnects 686 and/or the molded layer 688 via a thermal epoxy. In addition, in at least one example, as shown in the view provided in FIG. 6A, the device 600 can include processor 692 electrically coupled to the thermopile 680 and a memory component. In one example, the processor 692 can be electrically coupled to the thin film thermopile via the logic board 673. The memory component can store electronic instructions that, when executed by the processor 692, determine the core temperature of the user according to the algorithms and methods described above and elsewhere herein.

In at least one example, using the devices described herein, including the device 600 shown in FIGS. 6A-6C, a method of measuring core body temperature can include bonding a first junction 682 of a thin film thermopile 680 to a logic board 673 disposed in the device 600 and bonding a second junction 684 to the rear cover 616 of the device 600. In such an example, the first junction 682 can be the cold junction and the second junction 684 can be the hot junction measuring the temperature at the rear cover 616. These steps of the method are disclosed in at least FIGS. 6A-6C of the figures and described above. Another step of the method can include generating a heat flux correction factor based on a temperature difference ΔT, as shown in FIG. 5 and described above.

The method of measuring core body temperature can also include calculating the core body temperature of a user donning the device 600 based on an algorithm, described above with reference to FIG. 5, taking into account the heat flux correction factor. Using the devices described herein, including device 600 shown in FIGS. 6A-6C, another step of the method can include pressing the rear cover against a body before generating the heat flux correction factor. Also, at least as described above with reference to FIG. 5, the algorithm can correlate the temperature of the rear cover to as surface temperature of the body of the user, for example at the surface of the user's wrist 401, and the algorithm can correlate the surface temperature of the wrist 401 to the core body temperature.

Any of the features, components, and/or parts, including the arrangements and configurations thereof shown in FIGS. 6A-6C can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 6A-6C.

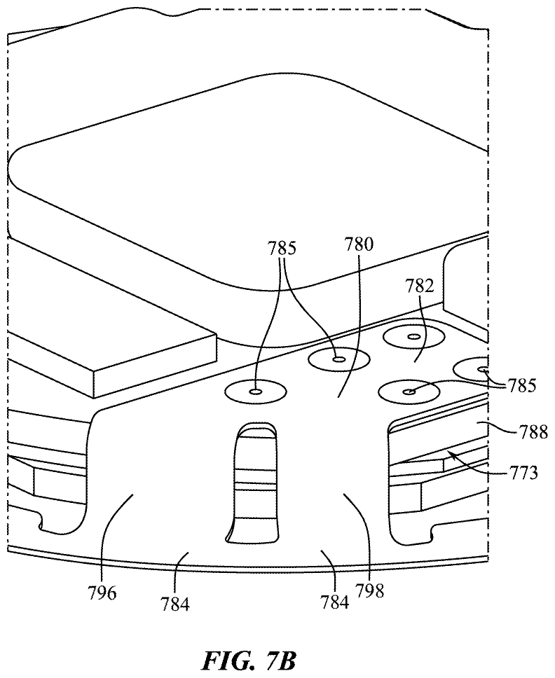

FIGS. 7A and 7B illustrate a partial top plan view and a partial top perspective view, respectively, of an example of a device 700 for measuring core body temperature. In the illustrated example of FIGS. 7A and 7B, at least a portion of the thermopile 780, which includes a cold junction 782 bonded to a logic board 773 and a hot junction 784 bonded to a rear cover 716, can be routed through a flex 794. FIG. 7A illustrates a component 799 disposed over at least a portion of the flex 794, for example over a lower portion including the cold junction 784 of the thermopile 780 disposed in the flex 794. The partial top perspective view of FIG. 7B shows the device 700 without the component 799. The component 799 can include any number of other components of the device 700, including but not limited to other flexes, brackets, antennas, and so forth.

In at least one example, the flex 794 can include a first arm 796 and a second arm 798. The first arm 796 can encompass the third portion (e.g., the third portion 680c shown in the thermopile 680 of FIG. 6) or hot junction 784. The second arm 798 of the flex 794 can encompass one or more other electrical flexes or thermopiles. In at least one example, the device 700 can include a number of hotbar pads 785 corresponding to electrical connections or vias extending through an upper molded layer to electrically couple the thermopile 780 to the underlying logic board via solder or another electrical connection. One or more of the hotbar pads 785 can be coupled with the thermopile 780 extending in the first arm 796 of the flex 764. One or more of the other hotbar pads 785 can be coupled with the other flex or component within the second arm 798 of the flex 794.

Any of the features, components, and/or parts, including the arrangements and configurations thereof shown in FIGS. 7A and 7B can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 7A and 7B.

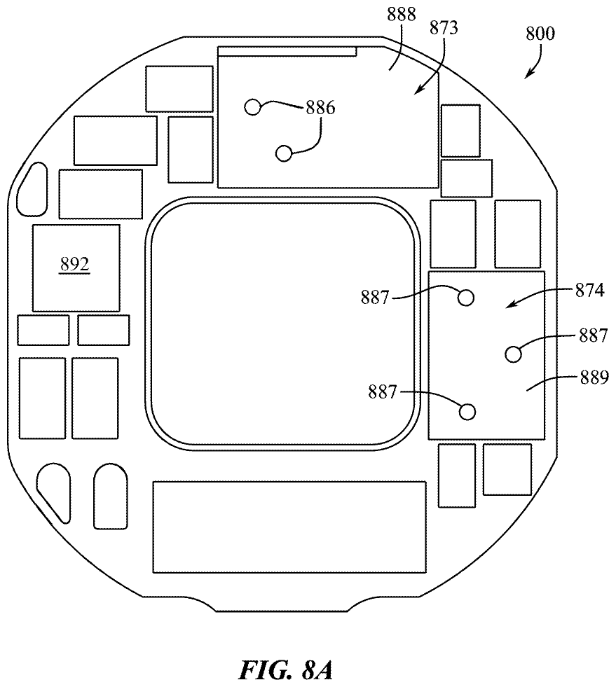

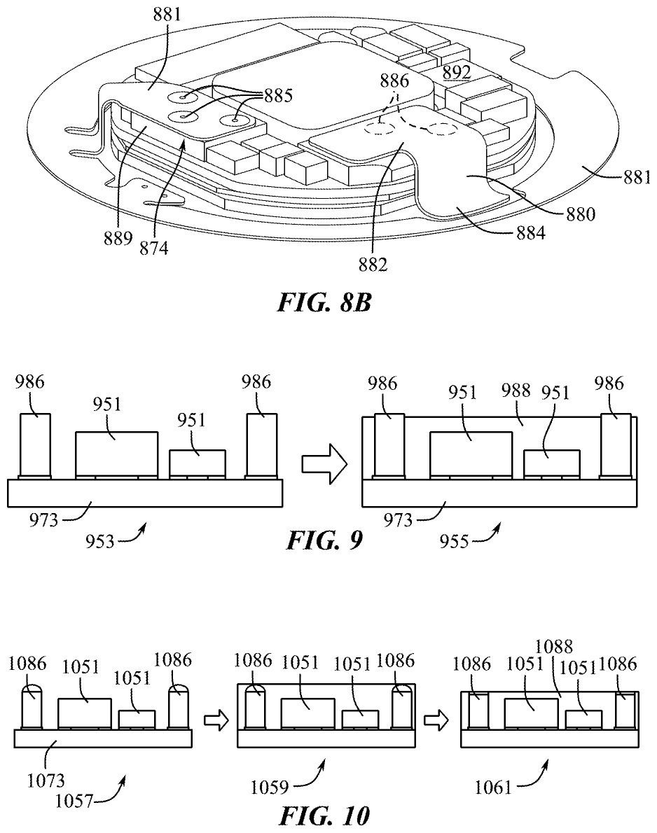

FIGS. 8A and 8B illustrate a top view and a perspective view, respectively, of another example of a device 800 including core measurement capabilities. In particular, the device 800 includes a processor 892 and a logic board 873 including an upper molded layer 888. One or more electrical connections or “vias” 886 can extend through the molded layer 888 to make contact with a cold junction 882, electrically coupling the thermopile to the logic board 873. FIG. 8A does not show the thermopile, in order to visualize the vias 886 and the upper molded layer 888 of the logic board 873.

FIG. 8B does illustrate the thermopile 880 with a cold junction 882 thereof bonded at least to the vias 886, shown in dotted lines representing the vias 886 underneath the cold junction 882 of the thermopile 880. The thermopile 880 can extend outward from the vias 886 and downward toward the hot junction 884. The hot junction 884 can be bonded directly to a rear cover (not shown) of the device 800. In at least one example, the thermopile 880 can include a rigid flex.

In addition, FIG. 8A illustrates a second logic board 874 with an upper molded layer 889 disposed thereon. One or more electrical vias 887 extending through the upper molded layer 889 of the logic board 874 can electrically couple to one or more other thermopiles or electrical flexes to the logic board 874 below the upper molded layer 889 thereof. FIG. 8B shows a flex 881 and hotbar pads 885 electrically coupled to the logic board 874 through the upper molded layer 889 thereof. The flex 881 can extend from the logic board 874 and down into a ring configuration surrounding a perimeter of the portion of the device 800 shown in FIG. 8B. In at least one example, the ring portion of the flex 881 can extend radially outward from and across the cold junction 882 of the thermopile 880 as shown.

Any of the features, components, and/or parts, including the arrangements and configurations thereof shown in FIGS. 8A and 8B can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 8A and 8B.

FIGS. 9-11 illustrate various manufacturing methods for forming a logic board or PCB with an upper molded layer and electrical conduction pins extending through the upper molded layer. For example, FIG. 9 illustrates a first step 953 of bonding conduction pins 986 to the PCB 973. Other components 951 of the PCB 973 can also be bonded to the PCB 973 as needed before the molded layer 988 is added. The components 951 and conduction pins 986 can be mounted using any known surface mounting techniques. A second step 955 can include adding the upper molded layer 988 over the components 951 and around the conduction pins 986 such that the conduction pins 986 extend through and flush with or above the upper surface of the molded layer 988. In one example, the molded layer 988 can be added using film assisted molding or other molding processes.

FIG. 10 shows another example of a method of manufacturing a logic board or PCB with an upper molded layer and electrical conduction pins extending through the upper molded layer. A first step 1057 of bonding conduction pins 1086 to the PCB 1073. Other components 1051 of the PCB 1073 can also be bonded to the PCB 1073 as needed before the molded layer is added. The components 1051 and conduction pins 1086 can be mounted using any known surface mounting techniques.

A second step 1059 can include regular molding of the upper molded layer 1088 to the PCB 1073 around the conduction pins 1086 and components 10751 on the PCB 1073. A third step 1061 according to the example shown in FIG. 10 can include grinding or laser cutting/etching a top portion or layer of the upper molded layer 1088 until top portions of the conduction pins 1086 are exposed, as shown. In at least one example, a portion of the conduction pins 1086 can be ground or laser etched flush with the upper surface of the upper molded layer 1088.

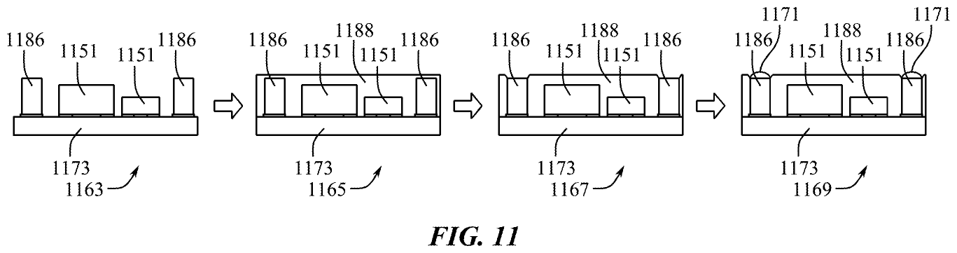

FIG. 11 shows another example of a method of manufacturing a logic board or PCB with an upper molded layer and electrical conduction pins extending through the upper molded layer. A first step 1163 of bonding conduction pins 1186 to the PCB 1173. Other components 1151 of the PCB 1173 can also be bonded to the PCB 1073 as needed before the molded layer is added. The components 1151 and conduction pins 1186 can be mounted using any known surface mounting techniques. A second step 1165 can include regular molding of the upper molded layer 1088 to the PCB 1073 around the conduction pins 1086 and components 1073 on the PCB 1073.

A third step 1167 of the example of FIG. 11 can include laser cutting or etching through the upper molded layer 1188 to expose the conduction pins 1186. A fourth step 1169 can include a ball drop step of adding electrically conductive material balls or drops 1171 to the top of the vias 1186.

Any of the steps, methods, features, components, and/or parts, including the arrangements and configurations thereof shown in FIGS. 9-11 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, steps, methods, features, components, and parts shown in FIGS. 9-11.

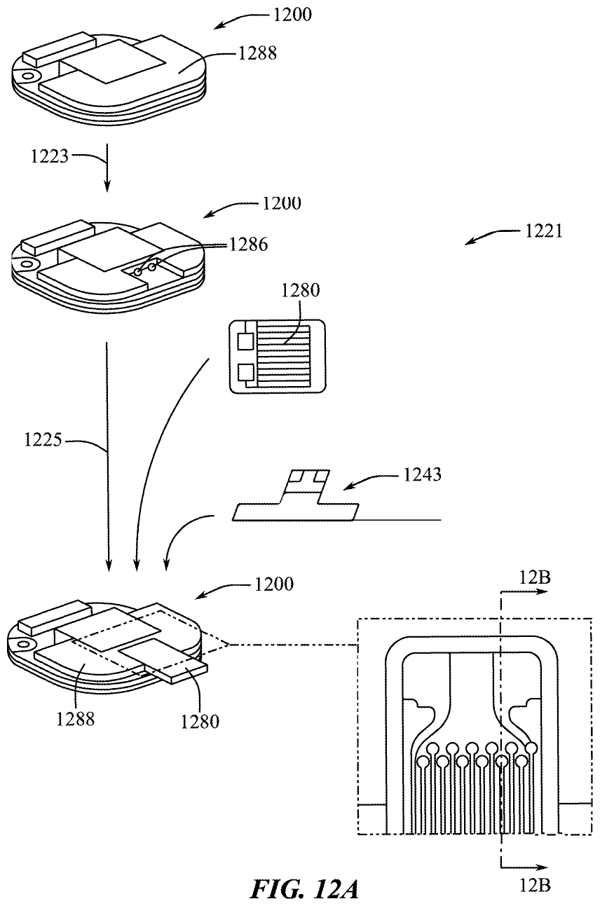

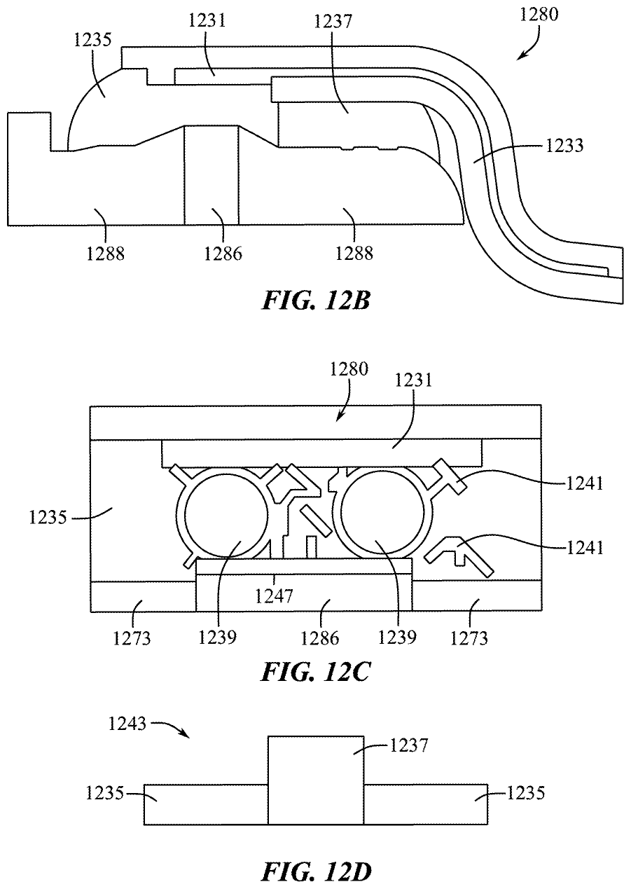

FIGS. 12A-12D illustrate an example of a thermopile integration into a device and/or onto the logic boards and PCBs disclosed herein. FIG. 12A shows a process flow of the integration process 1221, including the manufacturing and assembly of a portion of an electronic device configured to measure core temperature, according to the present disclosure. FIGS. 12B-12D illustrate various aspects of the integrated thermopile coupled to the PCB. FIG. 12A shows a portion of an example of a device 1200, including an upper molded layer 1288 of a PCB 1273. In at least one example, a first step 1223 of assembly or manufacturing can include laser ablation of a portion of the upper molded layer 1288. The ablation in this step 1223 can expose one or more electrical connection conduction pins 1286. A next step 1225 can include bonding a thermopile 1280 to the conduction pins 1286 using an isotropic conductive film (ICF)/non-conductive film (NCF) assembly (ICF/NCF assembly) 1243. The ICF/NCF assembly 1243 can be used to bond the thermopile 1280 to the exposed conduction pins 1286.

FIG. 12B shows a partial cross-sectional view of the assembled device 1200 as indicated in FIG. 12A. FIG. 12B shows a finished thermopile integration, including a thermopile 1280 having a flex spring 1245 and a CVL component 1233 on either side of a copper portion 1231. The flex spring 1245 can be die cut to maximize an area pushing back against spring back force and delamination of the thermopile 1280. The copper portion 1231 of the thermopile 1280 shown in FIG. 12B can be part of a cold junction for detecting temperature differences between the location of the cold junction and the hot junction disposed against a rear cover of the device. In addition, FIG. 12B shows an upper molded layer 1288, which can be disposed on top of a PCB or logic board, as well as a conduction pin 1286 extending through the upper molded layer 1288.

In addition, an ICF portion 1235 can be used to bond the thermopile 1280 to the conduction pin 1286 to electrically connect at least the copper portion 1231 to the conduction pin 1286 as shown. NCF portion 1237 can also be used to bond the CVL component 1233 to the upper molded layer 1288. In at least one example, as shown in FIG. 12B, the upper surface of the upper molded layer 1288 can be rough or irregular due to the laser ablation of the upper molded layer 1288 performed to expose the conduction pin 1286, as discussed above with reference to FIG. 12A. The ICF and NCF portions 1235, 1237 can conform to the upper surface of the upper molded layer 1288 and form a sufficient bond to electrically couple the thermopile 1280 to the conduction pin 1286.

FIG. 12C shows another cross-sectional view of the thermopile integration shown in FIG. 12B. In at least one example, one or more silver coated copper elements 1239 can be integrated into the ICF portion 1235. The silver coated copper elements can form dendrites 1241 dispersed throughout the ICF portion 1235 to electrically connect the copper portion 1231 of the thermopile 1280 to the conduction pin 1286. In at least one example, solder 1247 can be disposed on top of the conduction pin 1286 to form a connection between the dendrites 1241 or other portions of the silver coated copper elements 1239 to the conduction pin 1286. In at least one example, the dendrites 1241 can extend through any oxidation layer on the conduction pin 1286 that may occur due to the laser ablation of the conduction pin 1286 during ablation of the upper molded layer 1288 to expose the conduction pin 1286.

As noted above, rough topography of the upper molded layer 1288 and conduction pin 1286 due to laser ablation can be accommodated by the ICF/NCF assembly 1243 and portions 1235, 1237 thereof. The ICF portion 1235 can be advantageous to use where lower bonding pressures but larger bonding areas are desired. As shown in FIG. 12D, a stepped ICF/NCF assembly 1243 can be used, with ICF portions 1235 adjacent to an NCF portion 1237, with the NCF portion 1237 extending higher than the ICF portions 1235. In at least one example, the NCF portion 1237 can extend at about 40 micrometers while the adjacent NCF portions 1235 can stand lower at about 30 micrometers. This stepped geometry of the ICF/NCF assembly 1243 can be used during manufacturing and assembly to accommodate the rough topography of the laser ablated upper molded layer 1288 and conduction pin 1286 to provide a sufficient bonding and electrical communication between the cold junction of the thermopile 1280 and the conduction pin 1286.

Any of the features, components, and/or parts, including the arrangements and configurations thereof shown in FIGS. 12A-12D can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 12A-12D.

To the extent applicable to the present technology, gathering and use of data available from various sources can be used to improve the delivery to users of invitational content or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, TWITTER® ID's, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver targeted content that is of greater interest to the user. Accordingly, use of such personal information data enables users to calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services or anytime thereafter. In another example, users can select not to provide mood-associated data for targeted content delivery services. In yet another example, users can select to limit the length of time mood-associated data is maintained or entirely prohibit the development of a baseline mood profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not target to be exhaustive or to limit the embodiments to the precise forms disclosed. It will be apparent to one of ordinary skill in the art that many modifications and variations are possible in view of the above teachings.

What is claimed is:

1. An electronic device, comprising:
a housing defining an internal volume, a front opening, and a rear opening;
a display component disposed at the front opening;
a rear cover disposed at the rear opening;
a logic board disposed in the internal volume; and
a thin film thermopile including a cold junction and a hot junction,
wherein:
the first junction is bonded to the logic board; and
the second junction is bonded to the rear cover.

2. The electronic device of claim 1, wherein:
the logic board includes an upper molded layer; and
the cold junction is bonded to the upper molded layer.

3. The electronic device of claim 2, wherein the logic board comprises an electronic interconnect extending through the molded layer and contacting the first junction.

4. The electronic device of claim 2, wherein the first junction is bonded via an isotropic conductive film (ICF).

5. The electronic device of claim 1, wherein the second junction is bonded via a thermal epoxy.

6. The electronic device of claim 1, wherein at least a portion of the thin film thermopile is routed within a flex.

7. The electronic device of claim 1, further comprising a processor electrically coupled to the thin film thermopile via the logic board, the processor configured to determine a core temperature of a user contacting the rear cover based on a temperature difference between the first junction and the second junction.

8. The electronic device of claim 7, wherein the temperature difference is used to generate a heat flux correction factor.

9. The electronic device of claim 8, wherein the processor executes an algorithm stored on a memory component, the algorithm taking into account the heat flux correction factor to determine the core temperature.

10. A wearable electronic device, comprising:
a housing sidewall defining:
an internal volume;
a first strap retention feature; and
a second strap retention feature opposite the first strap retention feature;
a rear cover; and
a core temperature sensing assembly, comprising:
a logic board disposed in the internal volume; and
a temperature sensor including a first junction bonded to the logic board and a second junction bonded to the rear cover.

11. The wearable electronic device of claim 10, wherein the temperature sensor is configured to sense a temperature difference between the first junction and the second junction.

12. The wearable electronic device of claim 11, wherein the temperature sensor comprises a thin film thermopile.

13. The wearable electronic device of claim 10, wherein the rear cover is configured to press against a body of a user when the user dons the wearable electronic device via a retention strap connected to the first strap retention feature and the second strap retention feature.

14. The wearable electronic device of claim 13, wherein the rear cover defines an external rear surface of the wearable electronic device.

15. The wearable electronic device of claim 14, further comprising a display assembly having a transparent cover, the transparent cover defining an external front surface of the wearable electronic device opposite the external rear surface.

16. A method of measuring a core body temperature with a wearable electronic device having a first junction of a thin film thermopile bonded to a logic board and a second junction of the thin film thermopile bonded to a rear cover, the method comprising:
generating a heat flux correction factor based on a temperature difference between the first junction and the second junction; and
calculating the core body temperature based on an algorithm taking into account the heat flux correction factor.

17. The method of claim 16, wherein the method is configured to generate the heat flux correction factor when the rear cover is pressed against a body.

18. The method of claim 17, wherein the second junction measures a temperature of the rear cover.

19. The method of claim 16, wherein the algorithm correlates a temperature of the rear cover to a surface temperature of the body.

20. The method of claim 19, wherein the algorithm correlates the surface temperature of the body to the core body temperature.
